(19) United States
(12) Patent Application Publication
(10) Pub. No.: US 2011/0086429 A1
(43) Pub. Date: Apr. 14, 2011

(54) METHODS AND DEVICES FOR DETECTING OZONE AND CARBONYL-CONTAINING COMPOUNDS

(75) Inventor: Shigehisa Uchiyama, Chiba City (JP)

(73) Assignee: SIGMA-ALDRICH CO., St. Louis, MO (US)

(21) Appl. No.: 12/810,905

(22) PCT Filed: Dec. 22, 2008
(86) PCT No.: PCT/US2008/088018

§ 371 Date: Dec. 6, 2010

Related U.S. Application Data
(60) Provisional application No. 61/017,377, filed on Dec. 28, 2007.

(51) Int. Cl. G01N 30/00; G01N 33/00

(52) U.S. Cl. 436/96; 436/106; 422/88

(57) ABSTRACT

Methods and devices are provided for detecting the presence of both ozone and carbonyl-containing compounds in air samples. Method and device for detecting the presence of ozone and carbonyl-containing compounds in an air sample, the method comprising: (a) contacting an air sample with an ozone-reactive adsorbent wherein if ozone is present in the air sample, the ozone reacts with the ozone-reactive adsorbent to form an aldehyde product; (b) contacting the air sample from Step (a) further with a carbonyl-reactive adsorbent wherein if a carbonyl-containing compound is present in the air sample, the carbonyl-containing compound reacts with the carbonyl-reactive adsorbent to form a first hydrazone product; (c) eluting with a solvent from the carbonyl-reactive adsorbent into the ozone-reactive adsorbent, wherein any aldehyde product of step (a) forms a second hydrazone product; and (d) analyzing eluate from Step (c) for presence of hydrazone products formed in Step (b) and Step (c).

### CROSS REFERENCE TO RELATED APPLICATION

This application claims the benefit of U.S. Provisional Application No. 61/017,377, filed on Dec. 28, 2007. The entire disclosure of the above application is incorporated herein by reference.

### FIELD

The present invention generally relates to methods and devices for detecting presence of ozone and carbonyl-containing compounds in air samples.

### BACKGROUND

This section provides background information related to the present disclosure which is not necessarily prior art.

Carbonyl-containing compounds, such as formaldehyde and acetaldehyde, are ubiquitous pollutants that are formed, for example, through oxidation of hydrocarbons by ozone in the troposphere or by the reaction between ozone and terpenoid in indoor air. Ozone in the troposphere is also a major pollutant produced by various sources including photochemical transformation of vehicle exhaust containing nitrogen oxides, carbon monoxide, and volatile organic compounds.

Hauser and Bradley report a spectrophotometric method for determination of ozone in the atmosphere in which atmospheric ozone reacts with 1,2-bis-(4-pyridyl)ethylene (BPE) in glacial acetic acid. Hauser, T. R. and Bradley D. W. Anal. Chem. 1966. 38:1529-1532.

For the measurement of carbonyls, 2,4-dinitrophenylhydrazine (DNPH) has been used in active and passive air sampling methods. Uchiyama, S. and Hasegawa, S. Atmos. Environ. 1999. 33:1999-2005.

Many previous attempts were made to measure ozone in ambient air. The potassium iodide method involved collecting samples in an alkaline potassium iodide solution and analyzing them by colorimetry after acidifying with sulfamic acid. However, instability of ozone samples collected in alkaline potassium iodide solution has been reported. Further, the nitrite-impregnated filter method has been reported for the measurement of ozone. Nitrite is oxidized with ozone and analyzed by ion chromatography. Based on the nitrite principle, OSHA Method ID-214 was developed as an active air sampling system. However, these methods have the disadvantage of being affected by other oxidizing agents.

When ambient atmospheric sampling is conducted at higher humidity, potassium iodide in the ozone scrubber is likely to be wetted by atmospheric moisture and carbonyl-containing compounds are trapped in the ozone scrubber. Moreover, potassium iodide dissolved by airborne moisture can migrate into the DNPH cartridge and react to form unknown compounds.

Therefore there is a need for a method and device for detecting the presence of both ozone and carbonyl-containing compounds in an air sample.

### SUMMARY

This section provides a general summary of the disclosure, and is not a comprehensive disclosure of its full scope or all of its features.

There is now provided an example method for detecting the presence of ozone and carbonyl-containing compounds in an air sample. The example method generally includes:
(a) contacting an air sample with an ozone-reactive adsorbent wherein if ozone is present in the air sample, the ozone reacts with the ozone-reactive adsorbent to form an aldehyde product;
(b) contacting the air sample from Step (a) further with a carbonyl-reactive adsorbent wherein if a carbonyl-containing compound is present in the air sample, the carbonyl-containing compound reacts with the carbonyl-reactive adsorbent to form a first hydrazone product;
(c) eluting with a solvent from the carbonyl-reactive adsorbent into the ozone-reactive adsorbent, wherein any aldehyde product of Step (a) forms a second hydrazone product; and
(d) analyzing eluate from Step (c) for presence of hydrazone products formed or formable in Step (b) and Step (c).

Another example method is provided for detecting the presence of ozone and carbonyl-containing compounds in an air sample. The example method generally includes:
(a) drawing the air sample into a device;
(b) contacting the air sample to a first bed comprising 1,2-bis(4-pyridyl)ethylene-coated silica particles, wherein ozone present in the air sample is trapped by reacting with the 1,2-bis(4-pyridyl)ethylene to form pyridine-4-aldehyde;
(c) contacting the air sample to a second bed comprising 2,4-dinitrophenylhydrazine-coated silica particles, wherein a carbonyl-containing compound present in the air sample is trapped by reacting with the 2,4-dinitrophenylhydrazine to form carbonyl-2,4-dinitrophenylhydrazone;
(d) eluting with a solvent from the second bed to the first bed, wherein excess 2,4-dinitrophenylhydrazine reacts with pyridine-4-aldehyde to form pyridine-4-aldehyde-2,4-dinitrophenylhydrazone; and
(e) analyzing the carbonyl-2,4-dinitrophenylhydrazone and pyridine-4-aldehyde-2,4-dinitrophenylhydrazone formed in steps (c) and (d).

There is further provided an example device for detecting the presence of ozone and carbonyl-containing compounds in an air sample. The example device generally includes a housing; means for drawing an air sample through the housing, wherein the air sample enters the housing through a first opening and exits the housing through a second opening; an ozone-reactive adsorbent disposed within the housing; and a carbonyl-reactive adsorbent disposed within the housing; wherein the ozone-reactive adsorbent and the carbonyl-reactive adsorbent are arranged within the housing such that at least a portion of the air sample drawn through the housing contacts the ozone-reactive adsorbent before the carbonyl-reactive adsorbent.

As another example, a device for detecting the presence of ozone and carbonyl-containing compounds in an air sample is also provided. The example device generally includes means for affecting a first mode of action and means for affecting a second mode of action, wherein the first mode and second mode co-act to detect the presence of ozone and carbonyl-containing compounds in the air sample. The first mode of action generally includes:
(a) contacting an air sample with an ozone-reactive adsorbent wherein if ozone is present in the air sample, the ozone reacts with the ozone-reactive adsorbent to form an aldehyde product; and
(b) contacting the air sample from Step (a) further with a carbonyl-reactive adsorbent wherein if a carbonyl-containing compound is present in the air sample, the carbonyl-containing compound reacts with the carbonyl-reactive adsorbent to form a first hydrazone product.
   And the second mode of action generally includes:
(c) eluting with a solvent from the carbonyl-reactive adsorbent into the ozone-reactive adsorbent, wherein any aldehyde product of Step (a) forms a second hydrazone product; and
(d) analyzing eluate from Step (c) for presence of hydrazone products formed in Step (b) and Step (c).

There is still further provided an example kit for detecting the presence of ozone and carbonyl-containing compounds in an air sample. The example kit generally includes:
(a) a cartridge for detecting the presence of ozone and carbonyl-containing compounds in an air sample, and
(b) a solvent for eluting derivatives of the ozone and carbonyl-containing compounds from the cartridge.

As another example, a device is provided for detecting the presence of ozone and carbonyl-containing compounds in an air sample. The example device generally includes a first housing and a second housing; means for drawing an air sample through the first and second housings; an ozone-reactive adsorbent disposed within the first housing; and a carbonyl-reactive adsorbent disposed within the second housing; wherein the air sample is drawn through the first housing and then through the second housing such that at least a portion of the air sample drawn through the first and second housings contacts the ozone-reactive adsorbent before the carbonyl-reactive adsorbent.

As still another example, a device is provided for detecting the presence of ozone and carbonyl-containing compounds in an air sample. The example device generally includes an ozone-reactive adsorbent and a carbonyl-reactive adsorbent. Means are provided for drawing an air sample through the ozone-reactive adsorbent and carbonyl-reactive adsorbent.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A, where the scale is not observed for the sake of clarity, is an elevation view of an example device for detecting the presence of ozone and carbonyl-containing compounds in an air sample;

FIG. 1B is an enlarged elevation view of the device of FIG. 1A with caps removed from end portions of the device;

FIG. 1C is an elevation view of the device of FIG. 1A with the caps removed from the end portions of the device and with the device shown in use in a second mode of action;

FIG. 2A, where the scale is not observed for the sake of clarity, is an elevation view of a first cartridge of another example device for use in detecting the presence of ozone and carbonyl-containing compounds in an air sample;

FIG. 2B, where the scale is not observed for sake of clarity, is an elevation view of a second cartridge of the example device for use in detecting the presence of ozone and carbonyl-containing compounds in an air sample;

FIG. 2C is an elevation view of the first cartridge of FIG. 2A and the second cartridge of FIG. 2B shown together for use in a first mode of action;

FIG. 3 is a graphical representation demonstrating the solubility of pyridine-4-aldehyde in mixtures of dimethyl sulfoxide (DMSO) and acetonitrile at 25° C.;

FIG. 4 is a graphical representation demonstrating UV-visible absorption spectra of pyridine-4-aldehyde 2,4-DNPhydrazone and lower aliphatic aldehyde DNPhydrazones in acetonitrile solution (20 μmol/L);

FIG. 5 is a graphical representation demonstrating chromatographic profiles of pyridine-4-aldehyde and other carbonyl 2,4-DNPhydrazones;

FIG. 6 is a graphical representation demonstrating reaction of pyridine-4-aldehyde and DNPH with time in extracted solutions containing various concentrations of phosphoric acid; and

FIG. 7 is a graphical representation demonstrating changes in aldehyde concentrations with time in eluates from BPE (upper) and DNPH cartridges (lower).

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprise,” “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In various aspects of the invention, methods, devices, and kits are provided for detecting the presence of ozone and carbonyl-containing compounds in air samples.

As used herein, the term “carbonyl-containing compound” refers to a compound containing at least one carbonyl group, such as an aldehyde or ketone. Examples of such compounds include, without limitation, formaldehyde, acetaldehyde acetone, acrolein, propanal, 2-butanone, butanal, benzaldehyde, cyclohexanone, i-pentanal, pentanal, o-tolualdehyde, m-tolualdehyde, p-tolualdehyde, hexanal, 2,5-dimethylbenzaldehyde, heptanal, o-phthalaldehyde, octanal, nonanal and decanal. In a particular embodiment, the carbonyl-containing compound is formaldehyde, acetone and/or acetaldehyde.

Air samples used in the invention may be ambient or atmospheric air; or air collected from a pollutant source such as vehicle exhaust.

In one example embodiment, a method is provided for detecting the presence of ozone and carbonyl-containing compounds in an air sample. The example method generally includes:
(a) contacting an air sample with an ozone-reactive adsorbent wherein if ozone is present in the air sample, the ozone reacts with the ozone-reactive adsorbent to form an aldehyde product;
(b) contacting the air sample from Step (a) further with a carbonyl-reactive adsorbent wherein if a carbonyl-containing compound is present in the air sample, the carbonyl-containing compound reacts with the carbonyl-reactive adsorbent to form a first hydrazone product;
(c) (c) eluting with a solvent from the carbonyl-reactive adsorbent into the ozone-reactive adsorbent, wherein any aldehyde product of Step (a) forms a second hydrazone product; and
(d) analyzing eluate from Step (c) for presence of hydrazone products formed or formable in Step (b) and Step (c).

If any ozone is present in the air sample, the ozone-reactive absorbent acts to “trap” or bind the ozone. In one aspect, the ozone-reactive adsorbent is an inert support coated or “impregnated” with an ozone-reactive compound. Examples of such inert supports include silica particles, such as silica gel, alumina, styrene-divinylbenzene (XAD-2), Florisil®, glass beads and glass fiber filters. In a particular example embodiment, the inert support is silica. In a further particular example embodiment, the silica is in the form of silica gel, such as octadecyl silyl silica gel (C18).

Generally, the ozone-reactive compound can be any compound capable of reacting with ozone. Particularly, the ozone-reactive compound is an olefin-containing compound. In a particular example embodiment, the olefin-containing compound is a 1,2-disubstituted olefin that is substituted with electron-donating groups. For example, the olefin-containing compound can include, without limitation, 1,2-bis(4-pyridyl)ethylene (BPE), stilbene, 4,4′-dimethoxystilbene, 1,2-bis(2-pyridyl)ethylene, 4,4′-dinitrostilbene and 4,4-dinitrostilbene-2,2-disulfonic acid. The olefin-containing compound can undergo an ozonolysis reaction between the ozone and an ethylenic double bond of the olefin-containing compound to yield, inter alia, an aldehyde product. In a further particular example embodiment, the ozone-reactive compound is BPE and the aldehyde product formed is pyridine-4-aldehyde.

After at least a portion of the air sample contacts the ozone-reactive adsorbent, the air sample is then contacted with carbonyl-reactive adsorbent. If any carbonyl-containing compounds are present in the air sample, the carbonyl-reactive absorbent acts to “trap” or bind the carbonyl-containing compounds. In one aspect, the carbonyl-reactive adsorbent is an inert support coated or “impregnated” with a carbonyl-reactive compound. Examples of such inert supports include those employed to make the ozone-reactive adsorbent.

The carbonyl-reactive compound can generally be any compound capable of reacting with a carbonyl group, such as a ketone or aldehyde. Examples of carbonyl-reactive compounds include, without limitation, 2,4-dinitrophenylhydrazine (DNPH), 3-methyl-2-benzothiazolinonehydrazone (MBTH), O-(2,3,4,5,6-pentafluorobenzyl)hydroxylamine, O-benzylhydroxylamine, 2-diphenylacetyl-1,3-indandione-1-hydrazone, 5-dimethylaminonaphthalene-1-sulfohydrazide (dansyl-hydrazine), N-Methyl-4-hydrazino-7-nitrobenzofurazan, pentafluorophenylhydrazine and O-(4-cyano-2-ethoxybenzyl)hydroxylamine.

In a particular example embodiment, the carbonyl-reactive adsorbent can further include phosphoric acid, hydrochloric acid, sulfuric acid, or a combination thereof. For example, in one embodiment the carbonyl-reactive adsorbent may further include about 15 mmol phosphoric acid.

Subsequently, any carbonyl-containing compounds present in the air sample, will react with the carbonyl-reactive adsorbent to form a first hydrazone product. In a particular example embodiment, the carbonyl-reactive absorbent is DNPH and the first hydrazone product formed is carbonyl-2,4-dinitrophenylhydrazone (carbonyl-DNPH). In a further particular example embodiment, carbonyl-containing compounds which can form a first hydrazone product include, without limitation, formaldehyde, acetaldehyde and acetone.

Air contact with the ozone-reactive adsorbent substantially before the carbonyl-reactive adsorbent is ideal so that if any ozone is present in the air sample, the ozone will be substantially trapped in the ozone-reactive adsorbent and the ozone will not substantially interfere with trapping and detection of the carbonyl-containing compounds. In this case, the ozone-reactive adsorbent can be said to be acting as an “ozone scrubber”.

Following the air sample contacting the ozone-reactive adsorbent and the carbonyl-reactive adsorbent, any product formed is eluted or “extracted” with a solvent from the carbonyl-reactive adsorbent into the ozone-reactive adsorbent. In this elution step, excess DNPH from the carbonyl-reactive adsorbent is washed into the ozone-reactive adsorbent. If any aldehyde product was formed from ozone reacting with the ozone-reactive adsorbent, the excess DNPH reacts with the aldehyde product to form a second hydrazone product, i.e. an aldehyde-2,4-dinitrophenylhydrazone (aldehyde-DNPH). In a particular example, if pyridine-4-aldehyde was the product formed in the ozone-reactive adsorbent, then when excess DNPH is washed into the ozone-reactive adsorbent, the second hydrazone product formed is pyridine-4-aldehyde 2,4-dinitrophenylhydrazone (pyridine-4-aldehyde-DNPH).

The solvent employed for the elution step is a polar solvent. Examples of suitable solvents include, without limitation, acetonitrile, DMSO, or a solution of acetonitrile and DMSO. In a particular example embodiment, the solvent is a solution of acetonitrile containing from about 1% to about 99% DMSO corresponding to the amount of pyridine-4-aldehyde-DNPH formed. In yet a further example embodiment, the acetonitrile solution contains about 15% to about 50% DMSO.

After the elution step, the eluate is analyzed for the presence of hydrazone products formed, such as a carbonyl-DNPH (referred to as a first hydrazone product above) and aldehyde-DNPH (referred to as a second hydrazone product above) products. Analysis may comprise separating the hydrazone products and measuring concentration of the hydrazone products, such as by high performance liquid chromotagraphy (HPLC) or gas chromotagraphy (GC). In a particular example embodiment, HPLC is employed to analyze the hydrazone products formed and eluted during the method. This can allow for measurement of ozone and carbonyl-containing compounds, such as formaldehyde, acetaldehyde and acetone, in a single HPLC analysis.

In a further aspect of the method, the air sample is drawn through the ozone-reactive adsorbent followed by the carbonyl-reactive adsorbent by means for actively drawing the air sample such as, for example, an air pump, etc. The flow rate of air can be from about one ml/min for about 24 hours to about 2000 ml/min for about one hour. In a particular example embodiment, the air flow rate is from about 50 ml/min for about 24 hours to about 1000 ml/min for about one hour. Other suitable means for drawing the air sample through the ozone-reactive adsorbent and carbonyl-reactive adsorbent may be used within the scope of the present disclosure.

In yet a further aspect, the presence of ozone and carbonyl-containing compounds in an air sample is detected in a device. For example, a method of detecting the presence of ozone and carbonyl-containing compounds in an air sample is provided. The example method generally includes:
(a) drawing the air sample into a device;
(b) contacting the air sample to a first bed comprising 1,2-bis(4-pyridyl)ethylene-coated silica particles, wherein ozone present in the air sample is trapped by reacting with the 1,2-bis(4-pyridyl)ethylene to form pyridine-4-aldehyde;
(c) contacting the air sample to a second bed comprising 2,4-dinitrophenylhydrazine-coated silica particles, wherein a carbonyl-containing compound present in the air sample is trapped by reacting with the 2,4-dinitrophenylhydrazine to form carbonyl-2,4-dinitrophenylhydrazone;
(d) eluting with a solvent from the second bed to the first bed, wherein excess 2,4-dinitrophenylhydrazine reacts with pyridine-4-aldehyde to form pyridine-4-aldehyde-2,4-dinitrophenylhydrazone; and
(e) analyzing the carbonyl-2,4-dinitrophenylhydrazone and pyridine-4-aldehyde-2,4-dinitrophenylhydrazone formed in Steps (c) and (d).

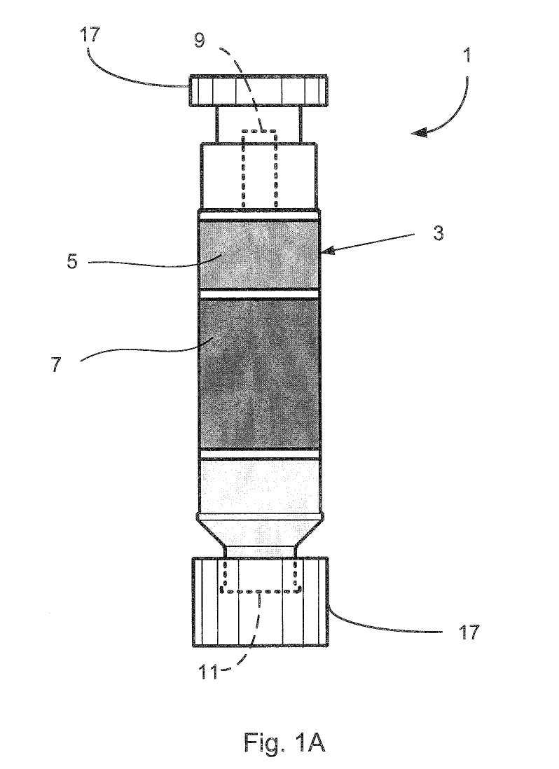

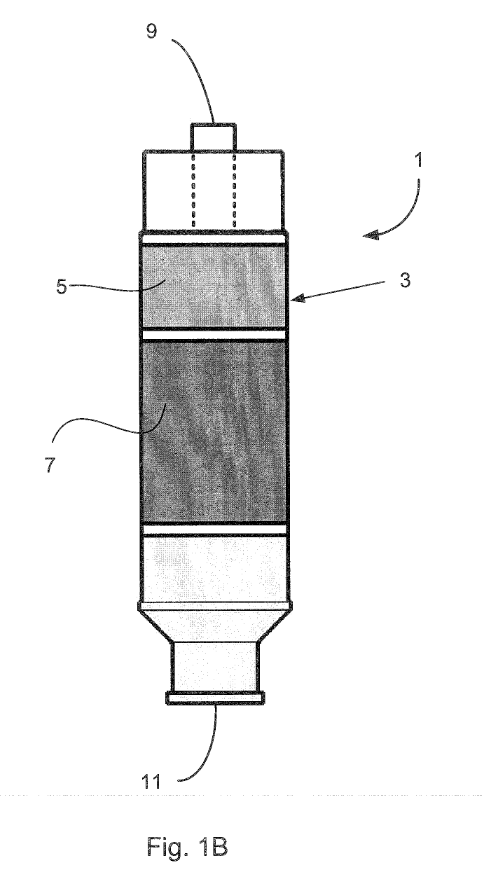

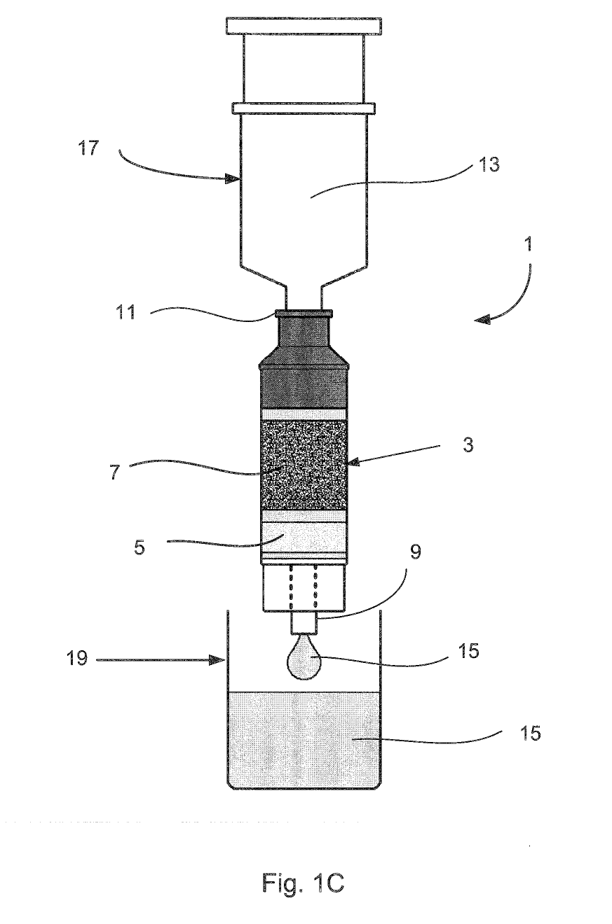

Therefore, in another example embodiment of the invention, a device 1 is provided for detecting the presence of ozone and carbonyl-containing compounds in an air sample. A general construction of the example device 1 is shown in FIGS. 1A-1C. The illustrated device 1 generally includes a housing 3, an ozone-reactive adsorbent 5 disposed within the housing 3, and a carbonyl-reactive adsorbent 7 disposed within the housing 3. Means can be provided for drawing, moving, etc. an air sample through the housing 3 such that the air sample enters the housing 3 through a first opening 9 and exits the housing 3 through a second opening 11. The ozone-reactive adsorbent 5 and the carbonyl-reactive adsorbent 7 are arranged within the housing 3 such that at least a portion of the air sample being drawn through the housing 3 contacts the ozone-reactive adsorbent 5 before the carbonyl-reactive adsorbent 7.

The housing 3 may be any inert structure such as a cartridge, a column, a syringe barrel, etc. The housing 3 may be made of any suitable inert material such as polyethylene, polypropylene, polytetrafluoroethylene, polyetheretherketone, and/or glass. The housing 3 can take any suitable shape for air sampling and elution. In a particular example embodiment, the housing 3 is a cylindrical polyethylene cartridge. Further, as depicted in FIG. 1A, when the device 1 is in a “stand by” mode, the device 1 may include one or more caps 17.

The ozone-reactive adsorbent 5 and the carbonyl-reactive adsorbent 7 are as substantially as described above. For example, the adsorbents 5 and/or 7 generally have a particle size from about 105 to about 210 μm; and/or an average particle size of about 150 μm. In the illustrated embodiment, both adsorbents 5 and 7 are disposed within the housing 3. As depicted in FIGS. 1A and 1B, the ozone-reactive adsorbent 5 and the carbonyl-reactive adsorbent 7 are arranged within the housing 3 such that at least a portion of air contacts the ozone-reactive adsorbent 5 before the carbonyl-reactive adsorbent 7. In a further particular example embodiment, the ozone-reactive adsorbent 5 and the carbonyl-reactive adsorbent 7 are present within the housing 3 in a ratio of about 1:3 respectively.

The means for drawing, moving, etc. an air sample (not depicted) through the housing 3 can be any suitable means for drawing, moving, conducting, etc. air, such as an air pump, etc. In a particular example embodiment, the means for drawing the air sample is attached to the second opening 11 of the housing 3. However, means for moving, pushing, etc. an air sample through the housing 3 may be attached to the first opening 9 of the housing 3 within the scope of the present disclosure.

As shown in FIG. 1C, the device 1 may further include means for introducing a solvent 13 into the housing 3 through the second opening 11 and means for collecting an eluate 15. The solvent 13 is substantially as described above. The means for introducing the solvent 13 into the housing 3 through the second opening 11 can be any suitable means such as a container, a funnel, bottle, tube, flagon, flask, glass, jar, jug, vial, etc. In FIG. 1C, container 17 is shown for use in introducing the solvent 13 into the housing 3. Further the means for collecting an eluate 15 can be any suitable means for collecting a liquid such as a container, a bottle, tube, flagon, flask, glass, jar, jug, vial, etc. In FIG. 1C, container 19 is shown for use in collecting eluate 15.

The device 1 may further comprise means for analyzing the eluate (not shown), such as an HPLC, GC, etc.

As a further example, the device 1 can also generally include means for affecting a first mode of action and means for affecting a second mode of action, wherein the first mode and second mode co-act to detect the presence of ozone and carbonyl-containing compounds in the air sample.

The first mode of action (FIG. 1B) is an air sampling mode which generally includes:
(a) contacting an air sample with the ozone-reactive adsorbent 5 wherein if ozone is present in the air sample, the ozone reacts with the ozone-reactive adsorbent 5 to form an aldehyde product; and
(b) contacting the air sample from Step (a) further with the carbonyl-reactive adsorbent 7 wherein if a carbonyl-containing compound is present in the air sample, the carbonyl-containing compound reacts with the carbonyl-reactive adsorbent 7 to form a first hydrazone product.

The first mode of action may further include utilizing means for drawing, moving, etc. the air sample to the ozone-reactive adsorbent 5 and the carbonyl-reactive adsorbent 7, such as an air pump, etc. In a particular example embodiment, the air drawing, moving, etc. means is attached to the second opening 11 of the housing 3 when the device 1 is in the air sampling mode (first mode of action).

The second mode of action (FIG. 1C) is an elution or “extraction” mode that generally includes:
(c) eluting with the solvent 13 (e.g., via container 17, etc.) from the carbonyl-reactive adsorbent 7 into the ozone-reactive adsorbent 5, wherein any aldehyde product of Step (a) from the first mode of action forms a second hydrazone product; and
(d) analyzing eluate 15 from Step (c) of this second mode of action for presence of hydrazone products formed in Step (b) of the first mode of action and Step (c) of this second mode of action.

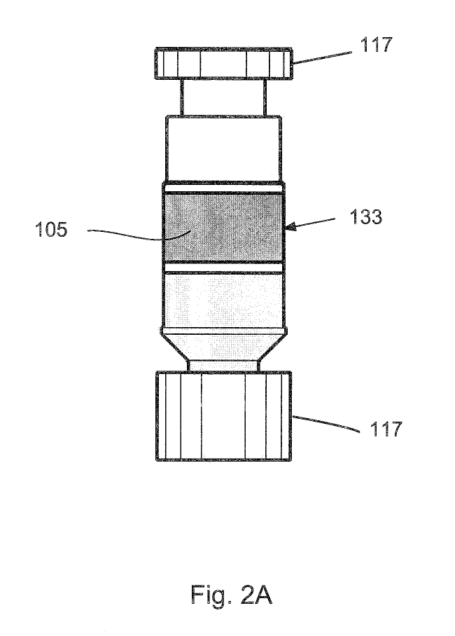

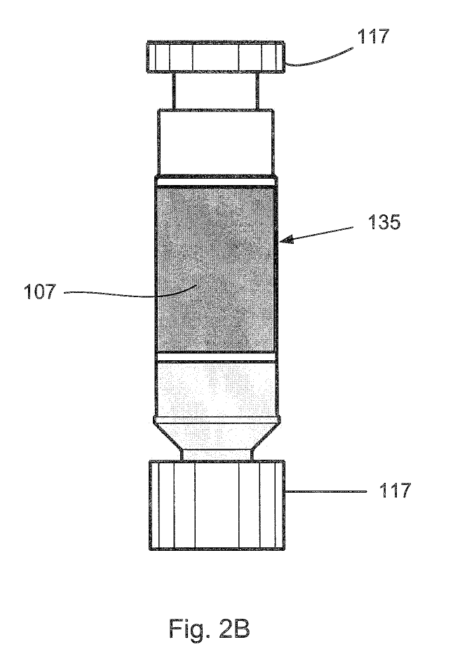

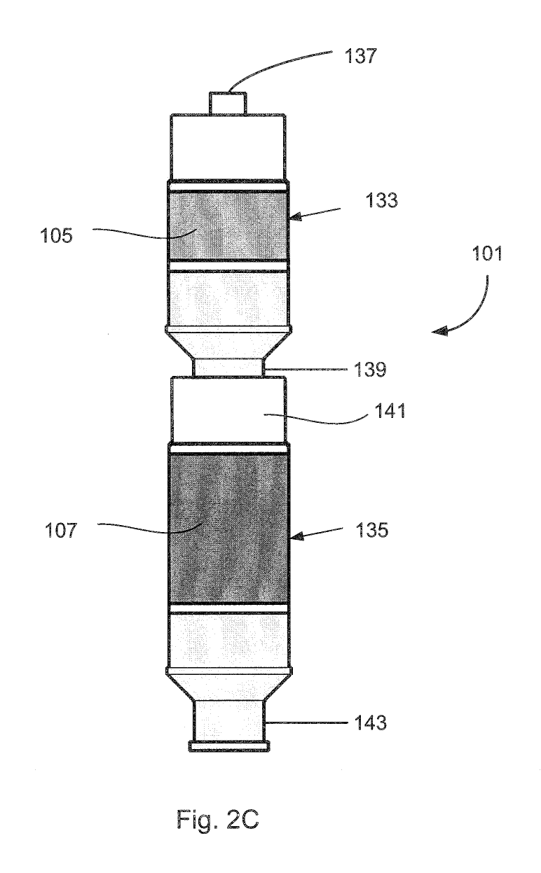

FIGS. 2A-2C illustrate another example device 101 (FIG. 2C) for detecting the presence of ozone and carbonyl-containing compounds in an air sample. The illustrated device 101 generally includes a first housing 133 and a second housing 135. In this embodiment, an ozone-reactive adsorbent 105 is disposed within the first housing 133, and a carbonyl-reactive adsorbent 107 is disposed within the second housing 135. Means can be provided for drawing, moving, etc. an air sample through the first housing 133 and through the second housing 135. As an example, at least a portion of the air sample drawn through the first and second housings 133 and 135 may contact the ozone-reactive adsorbent 105 before the carbonyl-reactive adsorbent 107. Alternative air flows may be provided within the scope of the present disclosure.

Each of the housings 133 and 135 may be any inert structure such as a cartridge, a column, a syringe barrel, etc. Each of the housings 133 and 135 may be made of any suitable inert material such as polyethylene, polypropylene, polytetrafluoroethylene, polyetheretherketone, and/or glass. Each of the housings 133 and 135 can take any suitable shape for air sampling and elution. In a particular example embodiment, the first housing 133 and the second housing 135 both include a cylindrical polyethylene cartridge. Further, as depicted in FIGS. 2A and 2B, when the device 101 is in a “stand by” mode, the housings 133 and 135 may each include one or more caps 117.

The ozone-reactive adsorbent 105 and the carbonyl-reactive adsorbent 107 of this embodiment are substantially as described above. For example, the adsorbents 105 and/or 107 generally have a particle size from about 105 to about 210 μm; and/or an average particle size of about 150 μm. In addition, in the illustrated embodiment, the ozone-reactive adsorbent 105 is disposed within the first housing 133 and the carbonyl-reactive adsorbent 107 is disposed within the second housing 135 in a ratio of about 1:3, respectively. Other ratios of adsorbents 105 and/or 107 may be used within the scope of the present disclosure.

The device 101 also generally includes means for affecting a first mode of action and means for affecting a second mode of action, wherein the first mode and second mode co-act to detect the presence of ozone and carbonyl-containing compounds in the air sample.

The first mode of action is an air sampling mode that generally includes:
(a) contacting an air sample with the ozone-reactive adsorbent 105 wherein if ozone is present in the air sample, the ozone reacts with the ozone-reactive adsorbent 105 to form an aldehyde product; and
(b) contacting the air sample with the carbonyl-reactive adsorbent 107 wherein if a carbonyl-containing compound is present in the air sample, the carbonyl-containing compound reacts with the carbonyl-reactive adsorbent 107 to form a first hydrazone product.

In the first mode of action of the illustrated embodiment (e.g., FIG. 2C, etc.), the first housing 133 is coupled to the second housing 135 (e.g., in series, with the first housing 133 positioned generally above the second housing 135, etc., as shown in the illustrated embodiment). More particularly in the illustrated embodiment, a second end portion 139 of the first housing 133 couples to a first end portion 141 of the second housing 135. Any suitable means may be used for coupling the first and second housings 133 and 135 together, for example, threaded connections, quick-release connections, press-fit connections, fasteners, etc. In other example embodiments, housings may be positioned differently than disclosed herein during modes of action. In still other example embodiments, intervening tubes, etc. may be provided between housings for use in coupling housings together.

The first mode of action may further include utilizing means for drawing, moving, etc. the air sample through the device 101. For example, the means can draw, move, push, etc. the air sample through the first housing 133 (and the ozone-reactive adsorbent 105 therein) and then through the second housing 135 (and the carbonyl-reactive adsorbent 107 therein) such that at least a portion of the air sample contacts the ozone-reactive adsorbent 105 within the first housing 133 before the carbonyl-reactive adsorbent 107 within the second housing 135 during sampling operation. The means may operate to move draw, move, push, etc. the air sample through the housings 133 and 135 differently than disclosed herein within the scope of the present disclosure. The means for drawing, moving, conducing, etc. the air sample can include any suitable means such as, for example, an air pump, etc. The means can, for example, be coupled to a second end portion 143 of the second housing 135 (e.g., to an opening defined by the second end portion 143 of the second housing 135, etc.). However, means for moving, pushing, etc. an air sample through the device 101 may be attached to a first end portion 137 of the first housing 133 (e.g., to an opening defined by the first end portion 137 of the first housing 133, etc.) within the scope of the present disclosure.

The second mode of action is an elution or “extraction” mode similar to that described in connection with device 1. The second mode of action generally includes:
(c) eluting with a solvent from the carbonyl-reactive adsorbent 107 into the ozone-reactive adsorbent 105, wherein any aldehyde product of Step (a) from the first mode of action forms a second hydrazone product; and
(d) analyzing eluate from Step (c) of this second mode of action for presence of hydrazone products.

In the second mode of action of the illustrated embodiment, for example, solvent can be eluted through the second housing 135 and then through the first housing 133. Device 101 may include means for introducing a solvent into the second housing 135 through its second end portion 143 and means for collecting an eluate through the first end portion 137 of the first housing 133. The solvent is substantially as described above. The means for introducing the solvent into the second housing 135 can be any suitable means such as a container, a funnel, bottle, tube, flagon, flask, glass, jar, jug, vial, etc. Further the means for collecting an eluate from the first housing 133 can be any suitable means for collecting a liquid such as a container, a bottle, tube, flagon, flask, glass, jar, jug, vial, etc. The solvent may move through the first and second housings 133 and 135 of the device 101 via gravity (e.g., the device may be oriented with the second housing 135 generally above the first housing 133, etc.), via pumps, etc. within the scope of the present disclosure.

The device 101 may further comprise means for analyzing the eluate (not shown), such as an HPLC, GC, etc.

In yet another example embodiment, a kit is provided generally including:
(a) a cartridge for detecting the presence of ozone and carbonyl-containing compounds in an air sample, and
(b) a solvent for eluting derivatives of the ozone and carbonyl-containing compounds from the cartridge.

The components of the kit can optionally be co-packaged, for example in a single container or in a plurality of containers within a single outer package, or co-presented in separate packaging (“common presentation”). As an example of co-packaging or common presentation, the kit may comprise, in a first container, the cartridge(s) for detecting the presence of ozone and carbonyl-containing compounds in an air sample, and, in a second container, the solvent for eluting derivatives of the ozone and carbonyl-containing compounds from the cartridge(s). For example, the first container may include a single cartridge containing ozone-reactive adsorbent and carbonyl-reactive adsorbent; or the first container may include one cartridge containing ozone-reactive adsorbent and another cartridge containing carbonyl-reactive adsorbent. In another example, the cartridge(s), and the solvent are separately packaged and available for sale independently of one another, but are co-marketed or co-promoted for use according to the invention.

### EXAMPLES

The following examples are merely illustrative, and do not limit this disclosure in any way.

### Example 1

Apparatus and Reagents. The HPLC system (Shimadzu, Kyoto, Japan) used included two LC-20AD pumps, an SIL-10AC autosampler and an SPD M20A photo-diode array detector. The analytical columns were 150 mm L×4.6 mm i.d. stainless steel tubes (Supelco Inc, Bellefonte, Pa., USA) packed with Ascentis RP-Amide, 3 μm particle size and Ascentis C18, 5 μm particle size. The mobile phase mixture was acetonitrile/water (40:60 v/v) containing 2 mmol/L ammonium acetate. The column temperature was 40° C. and the injection volume was 10 μL. Three air pumps (100 Dual GL Sciences Inc., Saitama, Japan) were used for the collection of air samples.

The water used in HPLC and sample preparation was deionized and purified using a Milli-Q Water System equipped with a UV lamp (Millipore, Bedford, Mass., USA). Acetonitrile was HPLC grade from Riedel-de Haen (AG, Seelze-Hannover, W. Germany). 2,4-Dinitrophenylhydrazine hydrochloride (>98%) was from Tokyo Kasei Co. Ltd. (Tokyo, Japan). Phosphoric acid (85% solution in water), hydrochloric acid (37%), trans-1,2-bis(4-pyridyl)ethylene (97%), pyridine-4-aldehyde (4-pyridinecarboxaldehyde, 97%) and ammonium acetate (99.999%) were from Sigma-Aldrich Inc., St. Louis, Mo., USA. Rezorian ozone scrubbers (3 mL/1.5 g potassium iodide) were from Supelco Inc. Silica gel (spherical, 105-210 μm particle size, 120 Å mean pore size) was from AGC Si-Tech. Co., Ltd. (Fukuoka, Japan).

Synthesis of pyridine-4-aldehyde-2,4-DNPhydrazone Hydrochloride Derivative. First, 2,4-dinitrophenylhydrazine hydrochloride (2.3 g) was dissolved in ethanol (400 mL) and hydrochloric acid (5 mL) was added. Pyridine-4-aldehyde (3 mL) was then added with continuous stirring. After a few minutes, the resulting precipitate was recovered by filtration and washed with water (3×500 mL), followed by ethanol (2×500 mL). Finally, the washed precipitate was dried for 3 hours at 105° C.

Preparation of DNPH-coated Silica Particles. Silica gel (50 g) was washed with acetonitrile (3×500 mL). To the washed silica gel was added a solution consisting of 2,4-Dinitrophenylhydrazine hydrochloride (0.25 g) and phosphoric acid (0.5 mL) dissolved in acetonitrile (500 mL). The mixture was stirred and the solvent was evaporated to dryness at 40° C. under vacuum using a rotary evaporator.

Preparation of BPE-coated Silica Particles. Silica gel (50 g) was washed with water (3×500 mL), methanol (2×500 mL) and acetonitrile (2×500 mL). To the washed silica gel was added trans-1,2-Bis(4-pyridyl)ethylene (BPE, 100 mg) in acetonitrile with continuous stirring. The solvent was then evaporated to dryness at 40° C. under vacuum using a rotary evaporator.

Preparation of BPE/DNPH Cartridge for Collection of Ozone and Carbonyls. BPE-coated silica particles (90 mg) and DNPH-coated silica particles (260 mg, containing 16 mmol phosphoric acid) were packed into a polyethylene cartridge (Rezorian tube, Supelco Inc, Bellefonte, Pa., USA) and stored in a refrigerator at 4° C. FIG. 1A-1C shows the schematic drawing of the BPE/DNPH cartridge and procedure for measuring ozone and carbonyls.

Air Sample Collection. The air sample was drawn through the cartridge from the BPE bed to the DNPH bed at a flow rate of 100 mL/min for 24 hours or 1000 mL/min for 1 hour. Ozone in the air sample was trapped in the BPE-coated silica bed and produced pyridine-4-aldehyde. Carbonyls in the air sample were trapped in the DNPH-coated silica bed and produced carbonyl 2,4-DNPhydrazones. The following chemistry was used to trap any ozone or carbonyl-containing compound present in the air sample (respectively):

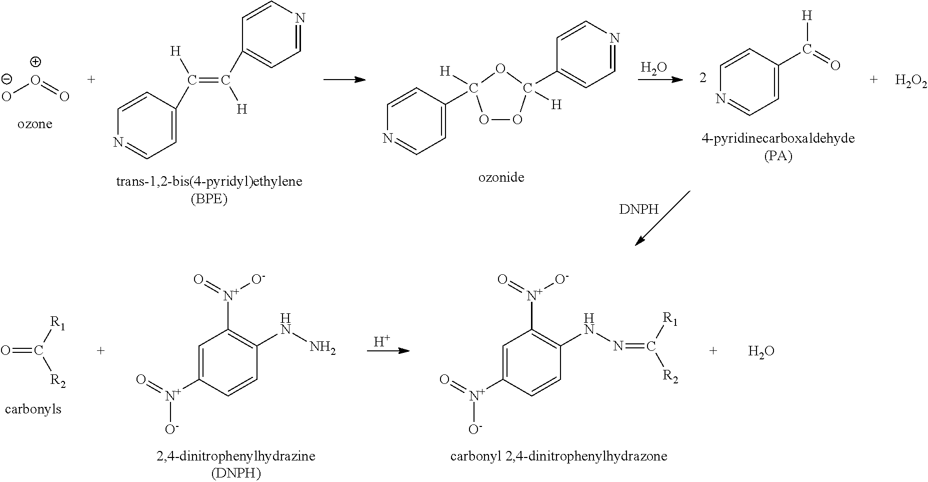

DNPH and carbonyl 2,4-DNPhydrazones in the second bed were not influenced by ozone because it was effectively trapped by reaction with BPE in the first bed.

Extraction and HPLC Analysis. Extraction was performed in the reverse direction to air sampling. Solvent passing through the BPE/DNPH cartridge washed DNPH into the BPE bed where it reacted with pyridine-4-aldehyde and formed the corresponding hydrazone derivative. The eluate from the BPE/DNPH-cartridge contained hydrazones derivatized with various carbonyls including pyridine-4-aldehyde formed from ozone.

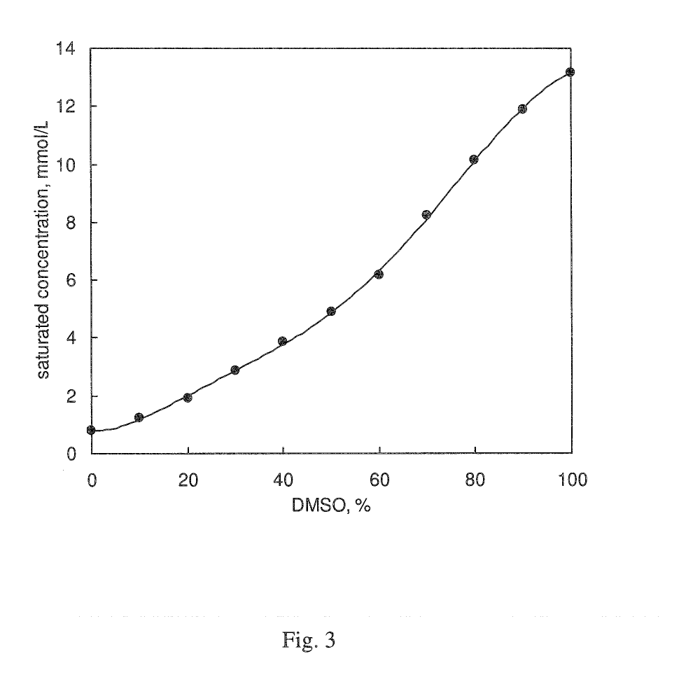

In general, acetonitrile is used as the eluent for carbonyl 2,4-DNPhydrazones, however pyridine-4-aldehyde 2,4-DNPhydrazone has poor solubility in acetonitrile. Therefore, DMSO was added to acetonitrile. Pyridine-4-aldehyde 2,4-DNPhydrazone dissolves in DMSO at relatively high concentration and FIG. 3 shows the solubility of pyridine-4-aldehyde in mixtures of dimethyl sulfoxide and acetonitrile at 25° C.

The solubility of pyridine-4-aldehyde in acetonitrile is about 0.79 mmol/L. If BPE in a BPE/DNPH cartridge reacts completely with ozone, about 0.66 mmol/L of pyridine-4-aldehyde are formed. Eluting with a solution of acetonitrile and DMSO helps to maintain high extraction efficiency. A solution containing 30% DMSO in acetonitrile was used as the eluent in this study.

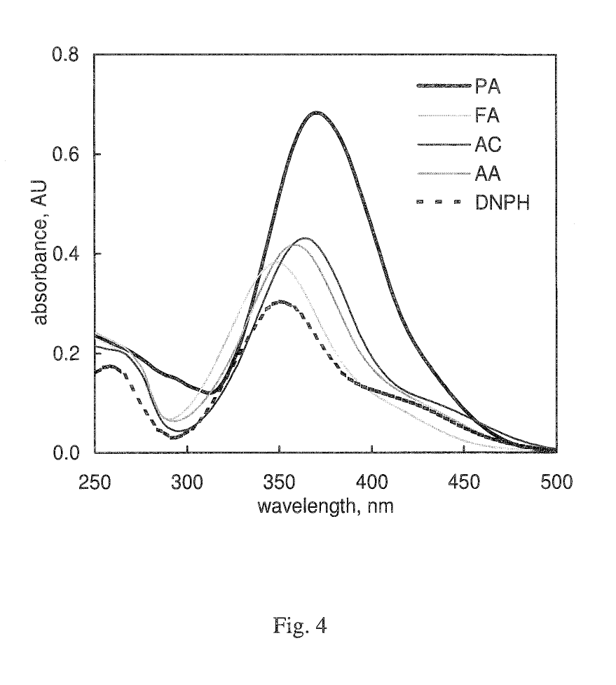

UV-visible absorption spectra of the pyridine-4-aldehyde 2,4-DNPhydrazone derivative (PA) are presented in FIG. 4. For reference, the absorption spectrum of DNPH, formaldehyde (FA), acetaldehyde (AA) and acetone (AC) derivatives are also shown. The spectral profiles of the pyridine-4-aldehyde-DNPhydrazone derivative are similar to those for lower aliphatic aldehyde-DNPhydrazones. As shown in Table 1, they exhibit long maximum absorption wavelengths (371 nm) and large absorption coefficients (3.4×10⁴ L/mol/cm).

TABLE 1
Maximum absorption wavelengths (λₘₐₓ) and molar
absorption coefficients (ε) of pyridine-4-aldehyde and C₁
-C₃ carbonyl-DNPH derivatives.

| | λₘₐₓ (nm) | ε (L/mol/cm) | mp (° C.) |
|---|---|---|---|
| pyridine-4-aldehyde-DNPhydrazide | 371 | 3.4 × 10⁴ | |
| DNPhydrazine | 351 | 1.5 × 10⁴ | ca. 200 ³⁰ |
| formaldehyde-DNPhydrazone | 349 | 1.9 × 10⁴ | 153-156 ³¹ |
| acetaldehyde-DNPhydrazone | 359 | 2.1 × 10⁴ | 165-168 ³¹ |
| acetone-DNPhydrazone | 360 | 2.1 × 10⁴ | |

Melting points of aldehyde-2,4-DNPhydrazones are literature values.

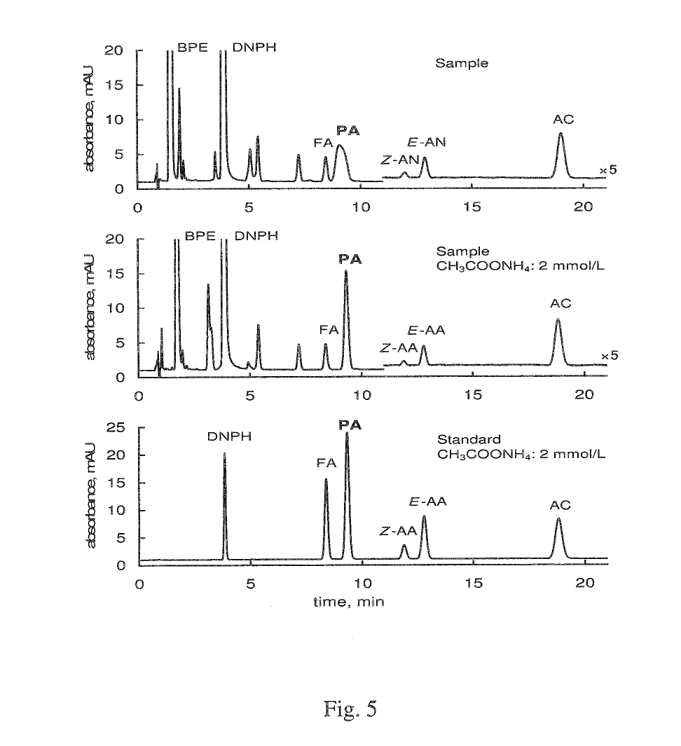

Analytical conditions for pyridine-4-aldehyde- and C₁-C₃-carbonyl-DNPH derivatives were examined using an Ascentis RP-Amide column. Two parallel air samplings were performed using the BPE/DNPH cartridge at 100 mL/min for 24 hours. The BPE/DNPH cartridges were subsequently extracted with 3 mL of mixed DMSO/ACN (30:70 v/v) solvent at 1 mL/min flow. After 24 h, the eluates were analyzed by HPLC using ACN/H₂O (40:60 v/v) mobile phases containing ammonium acetate (0, 0.1, 1, 2, 5, 10 or 20 mmol/L). FIG. 5 shows the chromatographic profiles of pyridine-4-aldehyde (PA), formaldehyde (FA), acetaldehyde (AA), and acetone (AC) 2,4-DNPhydrazones in the solutions extracted from air samplers and standard solutions.

The mobile phase mixture was acetonitrile/water (40:60 v/v) and acetonitrile/water (40:60 v/v) containing 2 mmol/L ammonium acetate. Mobile phase flow rate was 1.5 mL/min.

When the mobile phase contained no ammonium acetate, the pyridine-4-aldehyde 2,4-DNPhydrazone peak was extremely broad. The peak sharpened significantly when ammonium acetate was added to the mobile phase. Adding ammonium acetate in excess of 2 mmol/L to the mobile phase provided no further reduction in peak width. In the case of samples derived from standard solutions, the peak width did not change with the addition of ammonium acetate.

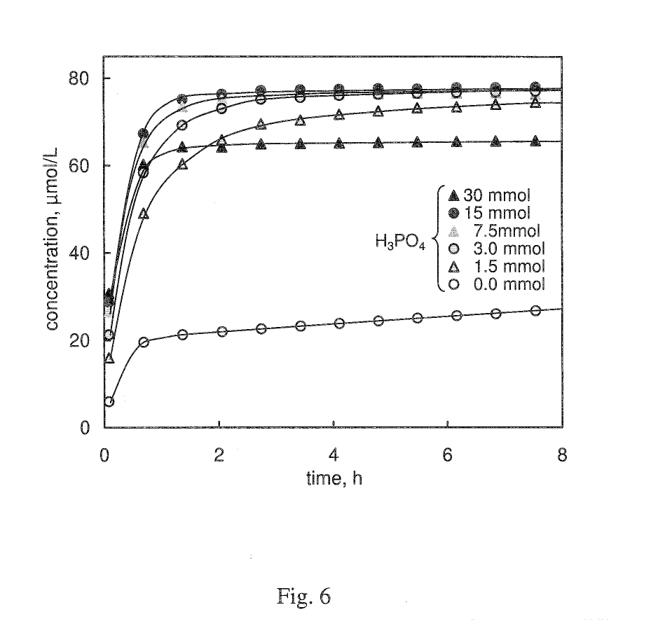

The reaction of pyridine-4-aldehyde and DNPH. Six parallel air samplings (100 mL/min for 24 hours) were performed using the BPE/DNPH cartridges made from DNPH-silica containing various phosphoric acid concentrations. The BPE/DNPH cartridges were subsequently extracted with 3 mL of DMSO/ACN (30:70 v/v) at 1 ml/min flow rate. The resulting hydrazone derivative mixtures were immediately analyzed by HPLC and the analyses were repeated at regular time intervals following storage at 25° C. FIG. 6 shows the concentration changes with time of pyridine-4-aldehyde DNPhydrazone in the extracted solutions containing 0, 1.5, 3.0, 7.5, 15 or 30 mmol phosphoric acid.

As the amount of phosphoric acid in the extraction solvent was increased, an obvious increase in the reaction rate was observed. The exception was when the extraction solution contained 30 mmol phosphoric acid. The reaction of pyridine-4-aldehyde and DNPH was completed in 4 hours in the presence of 15 mmol phosphoric acid. The facts that the derivatization reaction requires a catalytic amount of acid and hydrazone formation is reversible explains the behavior observed in FIG. 6. In exceedingly acidic aqueous solution (such as 30 mmol phosphoric acid), the hydrazone derivative is hydrolyzed back to the pyridine-4-aldehyde and DNPH. Thus, equilibrium is attained at a lower hydrazone concentration. FIG. 6 suggests that a suitable catalytic amount of phosphoric acid in DNPH-silica is about 15 mmol.

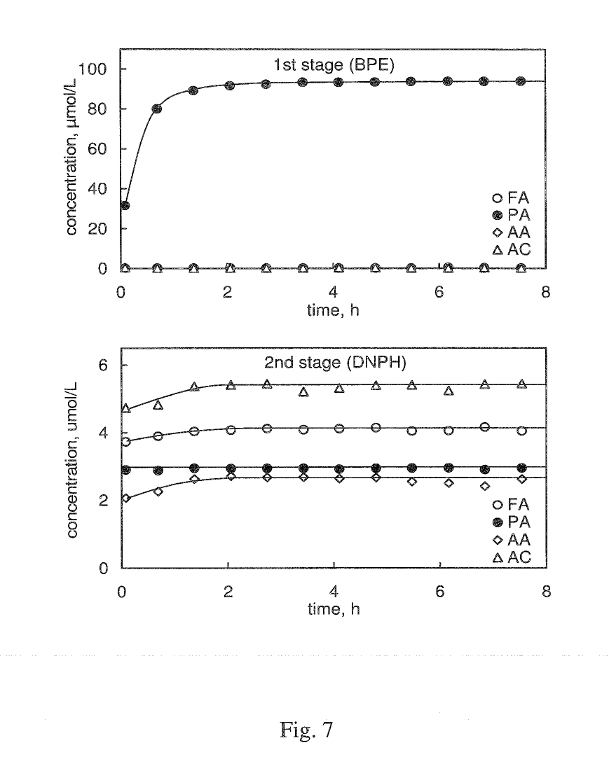

Separate BPE and DNPH cartridges were prepared and connected in series. An air sample was drawn through from BPE cartridge to the DNPH cartridge at 1000 mL/min for 2 hours. After collection, an unused DNPH cartridge was connected to the BPE cartridge and extraction from the DNPH cartridge with DMSO/ACN (30:70 v/v) at 1 mL/min was carried out. The sampled DNPH cartridge was extracted directly with DMSO/ACN (30:70 v/v) at 1 mL/min. FIG. 7 shows the concentration changes with time of aldehydes in the eluates from the BPE and DNPH sampling cartridges.

From the BPE cartridge, only pyridine-4-aldehyde was detected. All of the airborne carbonyls, and some pyridine-4-aldehyde, were detected from the DNPH cartridge. Thus, all of the airborne carbonyls (formaldehyde, acetaldehyde and acetone) passed through the BPE-cartridge and were trapped in the DNPH-cartridge. Nearly all (97%) of the pyridine-4-aldehyde formed by reaction of ozone with BPE remained in the BPE cartridge with 3% being detected in the DNPH cartridge.

Measurement of ambient air. Ambient air in Chiba city was collected using different DNPH cartridges. These included a DNPH cartridge alone, a DNPH cartridge coupled with a KI-ozone scrubbing cartridge and a two-bed BPE/DNPH cartridge. Air was sampled at 50 mL/min for 24 hours. The measured concentrations of ozone and carbonyls, together with the weather conditions, are listed in Table 2. June and July are the rainy season in Japan, which often leads to very high humidity. When sampling was performed during high humidity periods (July 4-5, July 12-13, July 17-18 and July 19-20), the concentrations of carbonyls collected with the KI-DNPH sampling train were lower compared to carbonyls collected with DNPH or BPE/DNPH cartridges. Without being bound by theory, we believe that carbonyls were trapped in the ozone scrubber as a consequence of the potassium iodide being wetted by atmospheric moisture. Moreover, it was observed that dissolved potassium iodide migrated into the DNPH cartridge and the yellow DNPH color changed to reddish brown. During lower humidity sampling periods (June 27-28, June 28-29 and July 21-22), the concentrations of carbonyls collected with the DNPH cartridge alone were lower when compared to the results from use of the KI-DNPH cartridge combination or the BPE/DNPH cartridge. Carbonyl-DNPhydrazones likely decomposed when the KI or BPE ozone-scrubbing agents were absent.

TABLE 2
Concentrations of ozone and carbonyls measured in ambient
air collected by a DNPH cartridge, a DNPH cartridge coupled
with a KI ozone scrubber and a BPE/DNPH cartridge.

| | ozone | formaldehyde | acetaldehyde | acetone |
|---|---|---|---|---|
| DNPH | | | | |
| June 27-28 | n.d. | 4.1 | 2.2 | 2.6 |
| June 28-29 | n.d. | 3.7 | 2.7 | 3.3 |
| July 4-5 | n.d. | 3.0 | 2.9 | 3.1 |
| July 12-13 | n.d. | 3.9 | 3.1 | 3.6 |
| July 17-18 | n.d. | 2.7 | 2.2 | 2.7 |
| July 19-20 | n.d. | 4.4 | 2.5 | 4.4 |
| July 21-22 | n.d. | 3.7 | 3.1 | 4.5 |
| KI-DNPH | | | | |
| June 27-28 | n.d. | 5.0 | 2.8 | 3.4 |
| June 28-29 | n.d. | 5.0 | 4.2 | 3.7 |
| July 4-5 | n.d. | 1.8 | 1.2 | 1.6 |
| July 12-13 | n.d. | 1.9 | 1.5 | 1.7 |
| July 17-18 | n.d. | 1.9 | 2.2 | 0.4 |
| July 19-20 | n.d. | 1.4 | 1.1 | 0.3 |
| July 21-22 | n.d. | 5.5 | 4.2 | 4.7 |
| BPE/DNPH | | | | |
| June 27-28 | 41.9 | 5.2 | 2.9 | 3.5 |
| June 28-29 | 43.8 | 5.3 | 4.0 | 4.8 |
| July 4-5 | 15.6 | 3.8 | 3.2 | 3.3 |
| July 12-13 | 7.1 | 4.3 | 3.0 | 4.0 |
| July 17-18 | 21.3 | 3.3 | 2.8 | 4.3 |
| July 19-20 | 6.9 | 4.2 | 2.4 | 4.5 |
| July 21-22 | 33.8 | 5.7 | 4.3 | 4.7 |

| weather conditions | Temp., ° C. | Humidity, % | weather |
|---|---|---|---|
| June 27-28 | 27.2 | 48.5 | clouds/fair |
| June 28-29 | 27.8 | 46.2 | fair/clouds |
| July 4-5 | 21.3 | 86.4 | rain |
| July 12-13 | 24.1 | 83.1 | clouds/rain |
| July 17-18 | 21.3 | 86.4 | rain |
| July 19-20 | 18.6 | 88.4 | clouds |
| July 21-22 | 27.0 | 42.5 | fair |

Air collections were performed simultaneously in Chiba city, June-July. Concentration units are in μg/m³.
n.d.: not detected

The use of a BPE/DNPH cartridge permits the detection of ozone and carbonyl-containing compounds in an air sample. A separate ozone-scrubbing cartridge is not necessary with the BPE/DNPH cartridge because the BPE performs this function. Moreover, air sampling can be performed effectively during either high or low humidity conditions.

All patents and publications cited herein are incorporated by reference into this application in their entirety.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

1. A method for detecting the presence of ozone and carbonyl-containing compounds in an air sample, the method comprising:
(a) contacting an air sample with an ozone-reactive adsorbent wherein if ozone is present in the air sample, the ozone reacts with the ozone-reactive adsorbent to form an aldehyde product;
(b) contacting the air sample from Step (a) further with a carbonyl-reactive adsorbent wherein if a carbonyl-containing compound is present in the air sample, the carbonyl-containing compound reacts with the carbonyl-reactive adsorbent to form a first hydrazone product;
(c) eluting with a solvent from the carbonyl-reactive adsorbent into the ozone-reactive adsorbent, wherein any aldehyde product of Step (a) forms a second hydrazone product; and
(d) analyzing eluate from Step (c) for presence of hydrazone products formed in Step (b) and Step (c).

2. The method of claim 1, wherein the ozone-reactive adsorbent is an inert support coated with an ozone-reactive compound.

3. The method of claim 2, wherein the ozone-reactive compound is an olefin-containing compound.

4. The method of claim 3, wherein the ozone-reactive compound is a 1,2-disubstituted olefin, wherein the 1,2-disubstituted olefin is substituted with electron-donating groups.

5. The method of claim 2 wherein the ozone-reactive compound is selected from the group consisting of 1,2-bis(4-pyridyl)ethylene, stilbene, 4,4′-dimethoxystilbene, 1,2-bis(2-pyridyl)ethylene, 4,4′-dinitrostilbene and 4,4-dinitrostilbene-2,2-disulfonic acid.

6. The method of claim 2, wherein the inert support is selected from the group consisting of silica gel, Florisil®, alumina, styrene-divinylbenzene, glass beads and glass fiber filters.

7. The method of claim 1, wherein the aldehyde product formed is pyridine-4-aldehyde.

8. The method of claim 1, wherein the carbonyl-reactive adsorbent is an inert support coated with a carbonyl-reactive compound selected from the group consisting of 2,4-dinitrophenylhydrazine, 3-methyl-2-benzothiazolinone hydrazone, O-(2,3,4,5,6-pentafluorobenzyl)hydroxylamine, O-benzylhydroxylamine, 2-diphenylacetyl-1,3-indandione-1-hydrazone, 5-dimethylaminonaphthalene-1-sulfohydrazide, N-Methyl-4-hydrazino-7-nitrobenzofurazan, pentafluorophenylhydrazine and O-(4-cyano-2-ethoxybenzyl)hydroxylamine.

9. The method of claim 8, wherein the inert support is selected from the group consisting of silica gel, Florisil®, alumina, styrene-divinylbenzene, glass beads and glass fiber filters.

10. The method of claim 1, wherein the first hydrazone product of Step (b) is carbonyl-2,4-dinitrophenylhydrazone.

11. The method of claim 8, wherein the carbonyl-reactive adsorbent further includes either phosphoric acid, hydrochloric acid, sulfuric acid, or a combination thereof.

12. The method of claim 1, wherein the solvent is a polar solvent.

13. The method of claim 12, wherein the solvent comprises a solution of acetonitrile and DMSO.

14. The method of claim 13, wherein the solvent comprises a solution of acetonitrile containing from about 15% to about 50% DMSO.

15. The method of claim 8, wherein eluting with a solvent washes excess carbonyl-reactive compound into the ozone-reactive adsorbent, which carbonyl-reactive compound reacts with any aldehyde product of Step (a) to form the second hydrazone product.

16. The method of claim 8 wherein the carbonyl-reactive compound is 2,4-dinitrophenylhydrazine, the aldehyde product is pyridine-4-aldehyde, and the second hydrazone product is pyridine-4-aldehyde 2,4-dinitrophenylhydrazone.

17. The method of claim 1, wherein analyzing eluate from Step (c) comprises separating the hydrazone products and measuring concentration of the hydrazone products formed in Step (b) and Step (c).

18. The method of claim 1, wherein the hydrazone products are separated and concentration measured by HPLC and/or GC.

19. The method of claim 17, wherein the hydrazone products are carbonyl-2,4-dinitrophenylhydrazone and pyridine-4-aldehyde-2,4-dinitrophenylhydrazone.

20. The method of claim 1, wherein the presence of ozone and carbonyl-containing compounds in an air sample is detected in a device.

21. The method of claim 20, wherein the air sample is drawn through the device from the ozone-reactive adsorbent to the carbonyl-reactive adsorbent.

22. The method of claim 21, wherein the air sample is actively drawn through the device at an air flow rate from about 1 ml/min for about 24 hours to about 2000 ml/min for about 1 hour.

23. The method of claim 22, wherein the air sample is actively drawn through the device at an air flow rate from about 50 ml/min for about 24 hours to about 1000 ml/min for about one hour.

24. The method of claim 20, wherein the air sample moves through the device from the ozone-reactive adsorbent to the carbonyl-reactive adsorbent.

25. The method of claim 24, wherein the ozone-reactive adsorbent and the carbonyl-reactive adsorbent are disposed within a housing.

26. The method of claim 24, wherein the ozone-reactive adsorbent is disposed within a first housing of the device and the carbonyl-reactive adsorbent is disposed within a second housing of the device.

27. A method of detecting the presence of ozone and carbonyl-containing compounds in an air sample, the method comprising:
(a) drawing the air sample into a device;
(b) contacting the air sample to a first bed comprising 1,2-bis(4-pyridyl)ethylene-coated silica particles, wherein ozone present in the air sample is trapped by reacting with the 1,2-bis(4-pyridyl)ethylene to form pyridine-4-aldehyde;
(c) contacting the air sample to a second bed comprising 2,4-dinitrophenylhydrazine-coated silica particles, wherein a carbonyl-containing compound present in the air sample is trapped by reacting with the 2,4-dinitrophenylhydrazine to form carbonyl-2,4-dinitrophenylhydrazone;
(d) eluting with a solvent from the second bed to the first bed, wherein excess 2,4-dinitrophenylhydrazine reacts with pyridine-4-aldehyde to form pyridine-4-aldehyde-2,4-dinitrophenylhydrazone; and
(e) analyzing the carbonyl-2,4-dinitrophenylhydrazone and pyridine-4-aldehyde-2,4-dinitrophenylhydrazone formed in steps (c) and (d).

28. A device for detecting the presence of ozone and carbonyl-containing compounds in an air sample, the device comprising:
a housing;
means for drawing an air sample through the housing, wherein the air sample enters the housing through a first opening and exits the housing through a second opening;
an ozone-reactive adsorbent disposed within the housing; and
a carbonyl-reactive adsorbent disposed within the housing;
wherein the ozone-reactive adsorbent and the carbonyl-reactive adsorbent are arranged within the housing such that at least a portion of the air sample drawn through the housing contacts the ozone-reactive adsorbent before the carbonyl-reactive adsorbent.

29. The device of claim 28, wherein the means for drawing an air sample through the housing is attached to the second opening.

30. The device of claim 28, further comprising means for introducing a solvent into the housing through the second opening.

31. The device of claim 28, further comprising means for collecting an eluate.

32. The device of claim 31, further comprising means for analyzing the eluate.

33. The device of claim 28, wherein the ozone-reactive adsorbent includes an inert support coated with an ozone-reactive compound.

34. The device of claim 33, wherein the ozone-reactive compound is an olefin-containing compound.

35. The device of claim 33, wherein the ozone-reactive compound is a 1,2-disubstituted olefin, wherein the 1,2-disubstituted olefin is substituted with electron-donating groups.

36. The device of claim 33, wherein the ozone-reactive compound is selected from the group consisting of 1,2-bis(4-pyridyl)ethylene, stilbene, 4,4′-dimethoxystilbene, 1,2-bis(2-pyridyl)ethylene, 4,4′-dinitrostilbene and 4,4-dinitrostilbene-2,2-disulfonic acid.

37. The device of claim 33, wherein the inert support is selected from the group consisting of silica gel, Florisil®, alumina, styrene-divinylbenzene, glass beads and glass fiber filters.

38. The device of claim 28, wherein the carbonyl-reactive adsorbent includes an inert support coated with a carbonyl-reactive compound selected from the group consisting of 2,4-dinitrophenylhydrazine, 3-methyl-2-benzothiazolinone hydrazone, O-(2,3,4,5,6-pentafluorobenzyl)hydroxylamine, O-benzylhydroxylamine, 2-diphenylacetyl-1,3-indandione-1-hydrazone, 5-dimethylaminonaphthalene-1-sulfohydrazide, N-Methyl-4-hydrazino-7-nitrobenzofurazan, pentafluorophenylhydrazine O-(4-cyano-2-ethoxybenzyl)hydroxylamine.

39. The device of claim 38, wherein the inert support is selected from the group consisting of silica gel, Florisil®, alumina, styrene-divinylbenzene, glass beads and glass fiber filters.

40. The device of claim 28, wherein the carbonyl-reactive adsorbent further includes either phosphoric acid, hydrochloric acid, sulfuric acid, or a combination thereof.

41. The device of claim 28, wherein the ozone-reactive adsorbent and the carbonyl-reactive adsorbent are present in a ratio of about 1:3 respectively.

42. A device for detecting the presence of ozone and carbonyl-containing compounds in an air sample, the device comprising means for affecting a first mode of action and means for affecting a second mode of action, wherein the first mode and second mode co-act to detect the presence of ozone and carbonyl-containing compounds in the air sample; wherein the first mode of action comprises:
(a) contacting an air sample with an ozone-reactive adsorbent wherein if ozone is present in the air sample, the ozone reacts with the ozone-reactive adsorbent to form an aldehyde product; and
(b) contacting the air sample from Step (a) further with a carbonyl-reactive adsorbent wherein if a carbonyl-containing compound is present in the air sample, the carbonyl-containing compound reacts with the carbonyl-reactive adsorbent to form a first hydrazone product;
and wherein the second mode of action comprises:
(c) eluting with a solvent from the carbonyl-reactive adsorbent into the ozone-reactive adsorbent, wherein any aldehyde product of Step (a) forms a second hydrazone product; and
(d) analyzing eluate from Step (c) for presence of hydrazone products formed in Step (b) and Step (c).

43. A kit for detecting the presence of ozone and carbonyl-containing compounds in an air sample comprising:
(a) a cartridge;
(b) an ozone-reactive adsorbent containing an ozone-reactive compound;
(c) a carbonyl-reactive adsorbent containing a carbonyl-reactive compound; wherein the carbonyl-reactive compound differs from the ozone-reactive compound; and
(d) a solvent for eluting derivatives of the ozone and carbonyl-containing compounds from the cartridge.

44. (canceled)

45. The kit of claim 43, wherein the cartridge contains the ozone-reactive adsorbent and the carbonyl-reactive adsorbent.

46. The kit of claim 43, comprising two or more cartridges, and wherein at least one cartridge contains the ozone-reactive adsorbent and at least another cartridge contains the carbonyl-reactive adsorbent.

47. A device for detecting the presence of ozone and carbonyl-containing compounds in an air sample, the device comprising:
a first housing and a second housing;
means for drawing an air sample through the first and second housings;
an ozone-reactive adsorbent disposed within the first housing; and
a carbonyl-reactive adsorbent disposed within the second housing;
wherein the air sample is drawn through the first housing and then through the second housing such that at least a portion of the air sample drawn through the first and second housings contacts the ozone-reactive adsorbent before the carbonyl-reactive adsorbent.

48. A device for detecting the presence of ozone and carbonyl-containing compounds in an air sample, the device comprising:
an ozone-reactive adsorbent containing an ozone-reactive compound;
a carbonyl-reactive adsorbent containing a carbonyl-reactive compound;
wherein the carbonyl-reactive compound differs from the ozone-reactive compound;
means for drawing an air sample through the ozone-reactive adsorbent and carbonyl-reactive adsorbent.

49. The device of claim 48, further comprising a cartridge, the ozone-reactive adsorbent and the carbonyl-reactive adsorbent being disposed within the cartridge.

50. The device of claim 48, further comprising first and second cartridges, the ozone-reactive adsorbent being disposed within the first cartridge and the carbonyl-reactive adsorbent being disposed within the second cartridge.

51. The device of claim 50, wherein the first cartridge is configured to be coupled to the second cartridge during operation of the device for detecting the presence of ozone and carbonyl-containing compounds in an air sample.

52. The device of claim 48, wherein the ozone-reactive adsorbent and the carbonyl-reactive adsorbent are oriented such that at least a portion of the air sample contacts the ozone-reactive adsorbent before the carbonyl-reactive adsorbent.
